(19) United States
(12) Patent Application Publication
(10) Pub. No.: US 2021/0282688 A1
(43) Pub. Date: Sep. 16, 2021

(54) MUSCULAR STRENGTH CHARACTERISTIC EVALUATION METHOD AND MUSCULAR STRENGTH CHARACTERISTIC EVALUATION DEVICE

(71) Applicant: Honda Motor Co., Ltd., Tokyo (JP)

(72) Inventors: Yasushi Ikeuchi, Saitama (JP); Toru Takenaka, Saitama (JP); Koji Akatsuka, Saitama (JP); Tomoyuki Shimono, Kanagawa (JP); Yu Goto, Kanagawa (JP); Mayu Miyake, Kanagawa (JP)

(73) Assignee: Honda Motor Co., Ltd., Tokyo (JP)

(21) Appl. No.: 17/197,061

(22) Filed: Mar. 10, 2021

(30) Foreign Application Priority Data
Mar. 11, 2020 (JP) 2020-041732

(51) Int. Cl. A61B 5/22

(52) U.S. Cl. CPC A61B 5/224; A61B 2560/04

(57) ABSTRACT

A muscular strength characteristic evaluation method evaluates a muscular strength characteristic of a limb 3 including a first rod L₁ having a base end supported by a first joint Ji, and a second rod L₂ supported by a free end of the first rod via a second joint J₂. The muscular strength characteristic evaluation method includes the following steps. In Step ST1, a free end of the second rod is moved at two or more different velocities vₐ, v_{b}, and v_{c} in a predetermined direction, and an output at the free end of the second rod is respectively measured at a predetermined position O. In Step ST2, a function indicating a relationship between the output and the velocity in a direction is calculated based on the output and the velocity. In Steps ST3 and ST4, the muscular strength characteristic is evaluated based on the function.

### CROSS-REFERENCE TO RELATED APPLICATION

This application claims the priority benefit of Japan application serial no. 2020-041732, filed on Mar. 11, 2020. The entirety of the above-mentioned patent application is hereby incorporated by reference herein and made a part of this specification.

### BACKGROUND

### Technical Field

The disclosure relates to a muscular strength characteristic evaluation method for evaluating a muscular strength characteristic of a limb of humans, animals, etc., and a muscular strength characteristic evaluation device for performing the muscular strength characteristic evaluation method.

### Related Art

As a model for evaluating muscles which contribute to movement in a two-dimensional plane of a limb including two joints such as an upper limb or a lower limb, the three-pair six-muscle group model classifies the muscles provided in a limb into a first antagonistic monoarticular muscle pair, a second antagonistic monoarticular muscle pair, and an antagonistic biarticular muscle pair (e.g., see Non-Patent Document 1: OSHIMA Toru, FUJIKAWA Tomohiko, and KUMAMOTO Minayori, “Functional Evaluation of Effective Muscular Strength Based on a Muscle Coordinate System Composed of Bi-articular and Mono-articular Muscles—Simplified Measurement Technique of Output Force Distribution”, Journal of Precision Engineering, Vol. 67, No. 6, p. 943-948 (2001)). In the three-pair six-muscle group model, the maximum output that can be exerted at the distal end of the limb is represented by a maximum output distribution in a hexagonal shape showing the sum of the maximum output of each muscle.

A muscular strength characteristic evaluation method is known to evaluate a muscular strength characteristic of a subject based on the three-pair six-muscle group model (e.g., see Patent Document 1: Japanese Patent Application Laid-Open No. 2000-210272). In Patent Document 1, a maximum output distribution is obtained based on outputs in predetermined four directions in the two-dimensional plane (four-point measurement method). Further, in Patent Document 1, the maximum output of each muscle is calculated based on the maximum output distribution, and the calculated maximum output of each muscle is used in muscular strength evaluation for rehabilitation and sports, training guidance evaluation, and the like.

It is known that there are two types of muscles including slow muscles and fast muscles. While slow muscles have a slower muscle contraction than fast muscles and have a weaker instantaneous exertion strength than fast muscles, slow muscles have better endurance than fast muscles. While fast muscles have a faster muscle contraction than slow muscles and have a stronger instantaneous exertion strength than slow muscles, fast muscles have weaker endurance than slow muscles. Slow muscles and fast muscles have different characteristics and functions, and their suitability ratio differs depending on the type of exercise. Therefore, it is important for athletes to evaluate the ratio of their slow muscles and fast muscles.

However, in the muscular strength characteristic evaluation method described in Patent Document 1, it is difficult to evaluate the ratio of fast muscles and slow muscles because only the maximum output distribution of muscular strengths can be obtained.

### SUMMARY

One aspect of the disclosure provides a muscular strength characteristic evaluation method, which evaluates a muscular strength characteristic of a limb (3) including a first rod (L₁) having a base end supported by a first joint (J₁), and a second rod (L₂) supported by a free end of the first rod via a second joint (J₂). The muscular strength characteristic evaluation method includes a step (ST1) of moving a free end of the second rod at two or more different velocities (vₐ, v_{b}, and v_{c}) in a predetermined direction, and respectively measuring an output at the free end of the second rod at a predetermined position (O); a step (ST2) of calculating a function indicating a relationship between the output and the velocity in the direction based on the output and the velocity; and a step (ST3 and ST4) of evaluating the muscular strength characteristic based on the function.

One aspect of the disclosure provides a muscular strength characteristic evaluation device (10), which evaluates a muscular strength characteristic of a limb (3) including a first rod (L₁) having a base end supported by a first joint (J₁), and a second rod (L₂) supported by a free end of the first rod via a second joint (J₂). The muscular strength characteristic evaluation device includes an acquisition means (10A) and a calculation means (10B). The acquisition means respectively acquires an output of a free end of the second rod at two or more different velocities (vₐ, v_{b}, and v_{c}) in a predetermined direction at a predetermined position. The calculation means calculates a function indicating a relationship between the output and the velocity in the direction based on the output and the velocity.

### BRIEF DESCRIPTION OF THE DRAWINGS

FIG. 1 is an explanatory view of a three-pair six-muscle group model for an upper limb.

FIG. 2 is an explanatory view of a maximum output distribution at a distal end of the upper limb.

FIG. 3 is a perspective view of a muscular strength characteristic evaluation device.

FIG. 4 is a top view of the muscular strength characteristic evaluation device.

FIG. 5 is a flowchart of a muscular strength evaluation processing.

FIG. 6 is a flowchart of a forward measurement process.

FIG. 7 shows graphs respectively of (A) a relationship between a velocity component and a forward-direction component of a force when outputted in the forward direction, (B) a relationship between a velocity component and a rearward-direction component of a force when outputted in the rearward direction, (C) a relationship between a velocity component and a leftward-direction component of a force when outputted in the leftward direction, and (D) a relationship between a velocity component and a rightward-direction component of a force when outputted in the rightward direction.

FIG. 8 is a graph showing output points obtained by measurement and approximate straight lines.

FIG. 9 is a graph showing maximum output distributions at velocities of 0.1 m/s, 0.2 m/s, and 0.3 m/s calculated using the approximate straight lines.

FIG. 10 is an explanatory view showing a method of respectively determining (A) a vertex A, (B) a vertex B and a vertex F, and (C) a vertex C, a vertex D, and a vertex E in the four-point measurement method.

FIG. 11 is a graph showing calculation results of maximum effective muscular strengths at velocities of 0.1 m/s, 0.2 m/s, and 0.3 m/s, respectively.

### DESCRIPTION OF THE EMBODIMENTS

In view of the above background, it is an objective of the disclosure to provide a muscular strength characteristic evaluation method capable of performing muscular strength evaluation according to a velocity, and a muscular strength characteristic evaluation device for performing the muscular strength characteristic evaluation method.

In order to solve the above problems, one aspect of the disclosure provides a muscular strength characteristic evaluation method, which evaluates a muscular strength characteristic of a limb (3) including a first rod (L₁) having a base end supported by a first joint (J₁), and a second rod (L₂) supported by a free end of the first rod via a second joint (J₂). The muscular strength characteristic evaluation method includes a step (ST1) of moving a free end of the second rod at two or more different velocities (vₐ, v_{b}, and v_{c}) in a predetermined direction, and respectively measuring an output at the free end of the second rod at a predetermined position (O); a step (ST2) of calculating a function indicating a relationship between the output and the velocity in the direction based on the output and the velocity; and a step (ST3 and ST4) of evaluating the muscular strength characteristic based on the function.

According to this configuration, the outputs at the two or more different velocities are acquired, and the function indicating the relationship between the output and the velocity is calculated based on the relationship between the acquired velocity and output. Since the calculated function represents the velocity dependence of the output, it is possible to perform the muscle strength evaluation according to the velocity by using the function.

In the step of measuring the output, the direction is set to at least four different directions in a plane defined by the first rod and the second rod. In the step of calculating the function indicating the relationship between the output and the velocity, the function is calculated with respect to each of the directions. The step of evaluating the muscular strength characteristic includes a step of calculating the output in each of the directions at the predetermined velocity by using the function, and creating a maximum output distribution (Qₐ, Q_{b}, and Q_{c}) in a hexagonal shape corresponding to contributions of each muscle of a muscle group model including a first antagonistic monoarticular muscle pair (e₁ and f₁) straddling the first joint, a second antagonistic monoarticular muscle pair (e₂ and f₂) straddling the second joint, and an antagonistic biarticular muscle pair (e₃ and f₃) straddling the first joint and the second joint.

According to this configuration, the function indicating the relationship between the output and the velocity in the four predetermined directions is acquired. Thereby, the outputs in each direction and at each velocity in the four directions can be acquired. Therefore, the maximum output distribution at each velocity can be obtained based on the four-point measurement method, and the muscular strength evaluation according to the velocity can be performed based on the maximum output distribution at each velocity.

In the above aspect, the step of evaluating the muscular strength characteristic may further include a step of calculating a contribution amount of each muscle of the muscle group model from the maximum output distribution (ST4).

According to this configuration, since the contribution amount of each muscle of the muscle group model at each velocity is calculated, a muscle group model close to the actual muscular strength characteristic of the subject can be constructed. As a result, the muscles to be strengthened can be identified, which can be utilized in muscular strength evaluation for rehabilitation and sports.

In the above aspect, a linear function may be used as the function indicating the relationship between the output and the velocity.

According to this configuration, the output at each velocity can be easily acquired.

In the above aspect, the free end of the second rod may be moved at the two or more velocities by applying multiple resistance forces to the free end of the second rod.

According to this configuration, since the subject may move the free end of the second rod by his/her own will, it is possible to reduce the anxiety which may be caused to the subject at the time of muscular strength evaluation.

In order to solve the above problems, one aspect of the disclosure provides a muscular strength characteristic evaluation device (10), which evaluates a muscular strength characteristic of a limb (3) including a first rod (L₁) having a base end supported by a first joint (J₁), and a second rod (L₂) supported by a free end of the first rod via a second joint (J₂). The muscular strength characteristic evaluation device includes an acquisition means (10A) and a calculation means (10B). The acquisition means respectively acquires an output of a free end of the second rod at two or more different velocities (vₐ, v_{b}, and v_{c}) in a predetermined direction at a predetermined position. The calculation means calculates a function indicating a relationship between the output and the velocity in the direction based on the output and the velocity.

According to this configuration, the outputs at the two or more different velocities are acquired, and the function indicating the relationship between the output and the velocity is calculated based on the relationship between the acquired velocity and output. Since the calculated function represents the velocity dependence of the output, it is possible to perform the muscle strength evaluation according to the velocity by using the function.

In the above aspect, the calculation means may use a linear function as the function indicating the relationship between the output and the velocity.

According to this configuration, the output at each velocity can be easily acquired.

In the above aspect, the acquisition means includes a fixing part (12), a slide unit (15), a drive unit (16), a sensor (20), and a damper (18). The fixing part fixes a subject. The slide unit is fixed to the fixing part. The drive unit is provided on the slide unit to be slidable in the direction. The sensor is provided on the drive unit and detects the output. The damper is provided between the slide unit and the drive unit. A resistance force of the damper is variable so that the free end of the second rod is movable at the two or more velocities by applying multiple resistance forces to the free end of the second rod.

According to this configuration, the outputs at the different velocities can be measured by changing the resistance force of the damper.

According to the above configuration, it is possible to provide a muscular strength characteristic evaluation method capable of performing muscular strength evaluation according to the velocity, and a muscular strength characteristic evaluation device for performing the muscular strength characteristic evaluation method.

In the following, an embodiment in which a muscular strength characteristic evaluation method according to the disclosure is used to evaluate a muscular strength characteristic of an upper limb on the right side of a person will be described with reference to the drawings.

The muscular strength characteristic evaluation method is based on a known three-pair six-muscle model. The three-pair six-muscle model modeling muscles which contribute to an output at a distal end (a carpal joint portion and an ankle joint portion) of a limb in a two-dimensional movement of a limb including two joints (a shoulder joint and an elbow joint; a hip joint and a knee joint) such as an upper limb and a lower limb. In the following, first, the three-pair six-muscle model will be described as necessary for the disclosure.

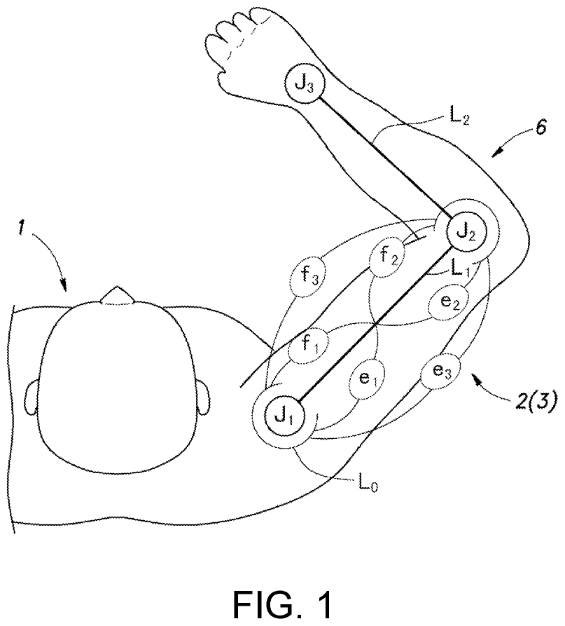

In the three-pair six-muscle model, as shown in FIG. 1, a limb 3 such as an upper limb 2 or a lower limb of a subject 1 is modeled as a two-joint link mechanism 6 including a first rod L₁ having a base end pivotally supported (supported) by a first joint J₁ at a base L₀, and a second rod L₂ pivotally supported (supported) by a free end of the first rod L₁ via a second joint J₂. More specifically, in the case where the upper limb 2 is modeled, the base L₀ corresponds to a scapula, the first joint J₁ corresponds to a shoulder joint, the first rod L₁ corresponds to a humerus, the second joint J₂ corresponds to an elbow joint, and the second rod L₂ corresponds to at least one of a radius and an ulna. Further, a free end J₃ of the second rod L₂ corresponds to a carpal joint portion. In the following, the free end J₃ of the second rod L₂ will be referred to as a limb distal end J₃.

The three-pair six-muscle group model, which models muscles contributing to movement in a two-dimensional plane including the first joint J₁, the second joint J₂, and the limb distal end J₃ of the limb 3, includes a first antagonistic monoarticular muscle pair f₁ and e₁ straddling the first joint J₁, a second antagonistic monoarticular muscle pair f₂ and e₂ straddling the second joint J₂, and an antagonistic biarticular muscle pair f₃ and e₃ straddling the first joint J₁ and the second joint J₂.

The first antagonistic monoarticular muscle pair f₁ and e₁ includes a muscle f₁ which bends the first joint J₁ and a muscle e₁ which extends the first joint J₁. The muscles f₁ and e₁ of the first antagonistic monoarticular muscle pair are attached to the base L₀ at one end and attached to the first rod L₁ at the other end, and are provided to straddle the first joint J₁. The first antagonistic monoarticular muscle f₁ corresponds to, for example, an anterior deltoid muscle, and the first antagonistic monoarticular muscle e₁ corresponds to, for example, a posterior deltoid muscle.

The second antagonistic monoarticular muscle pair f₂ and e₂ includes a muscle f₂ which bends the second joint J₂ and a muscle e₂ which extends the second joint J₂. The muscles f₂ and e₂ of the second antagonistic monoarticular muscle pair are attached to the first rod L₁ at one end and attached to the second rod L₂ at the other end, and are provided to straddle the second joint J₂. The second antagonistic monoarticular muscle f₂ corresponds to, for example, a brachialis muscle, and the second antagonistic monoarticular muscle e₂ corresponds to, for example, a triceps brachii muscle lateral head.

The antagonistic biarticular muscle pair f₃ and e₃ includes a muscle f₃ which simultaneously bends the first joint J₁ and the second joint J₂, and a muscle e₃ which simultaneously extends the first joint J₁ and the second joint J₂. The antagonistic biarticular muscle pair f₃ and e₃ is attached to the base L₀ at one end and attached to the second rod L₂ at the other end, and is provided to straddle the first joint J₁ and the second joint J₂, respectively. The antagonistic biarticular muscle f₃ corresponds to, for example, a biceps brachii muscle, and the antagonistic biarticular muscle e₃ corresponds to, for example, a triceps brachii muscle long head.

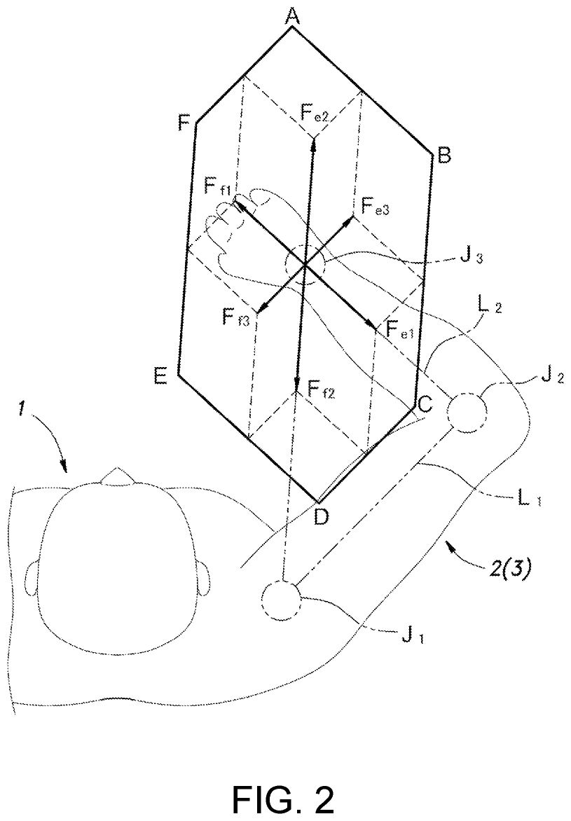

By the combination of outputs of the first antagonistic monoarticular muscle pair f₁ and e₁, the second antagonistic monoarticular muscle pair f₂ and e₂, and the antagonistic biarticular muscle pair f₃ and e₃, the magnitude and direction of the output at the limb distal end J₃ are determined. Setting a maximum output outputted by the first antagonistic monoarticular muscle f₁ to the limb distal end J₃ as F_{f1}, setting a maximum output outputted by the first antagonistic monoarticular muscle e₁ to the limb distal end J₃ as Fₑ₁, setting a maximum output outputted by the second antagonistic monoarticular muscle f₂ to the limb distal end J₃ as F_{f2}, setting a maximum output outputted by the second antagonistic monoarticular muscle e₂ to the limb distal end J₃ as Fₑ₂, setting a maximum output outputted by the antagonistic biarticular muscle f₃ to the limb distal end J₃ as F_{f3}, and setting a maximum output outputted by the antagonistic biarticular muscle e₃ to the limb distal end J₃ as Fₑ₃, as shown in FIG. 2, a distribution map of the maximum outputs (hereinafter referred to as “a maximum output distribution”) by these three-pair six-muscles obtained at the limb distal end J₃ is simply represented by a hexagon ABCDEF corresponding to the contribution of each muscle. The maximum output of each muscle (hereinafter referred to as “a functional effective muscular strength”) is a greatest force that each muscle can exert (output) and is represented by an in-plane vector defined by the first rod L₁ and the second rod L₂. While the details of the calculation method of the hexagon ABCDEF are omitted herein as they are conventionally known, reference may still be made to the above-mentioned Non-Patent Document 1.

In the hexagon ABCDEF, a side AB, a side DE, and the second rod L₂ are parallel to each other, and a side CD, a side FA, and the first rod L₁ are parallel to each other. Further, a side BC, a side EF, and a straight line connecting the limb distal end J₃ and the first joint J₁ are parallel to each other. An output F_{A} at a point A, an output FB at a point B, an output F_{C} at a point C, an output F_{D} at a point D, an output F_{E} at a point E, and an output F_{F} at a point F in FIG. 2 are represented by Formula (1) below. The functional effective muscular strength F_{f1}, F_{f2}, F_{f3}, Fₑ₁, Fₑ₂, and Fₑ₃ of the muscles may be calculated from the hexagon ABCDEF using Formula (1).

$$\begin{matrix}[ Mathematical Formula 1 ] & \\ \{ \begin{matrix}{F}_{A} = {F}_{f 1} + {F}_{e 2} + {F}_{e 3} \\ {F}_{B} = {F}_{e 1} + {F}_{e 2} + {F}_{e 3} \\ {F}_{C} = {F}_{e 1} + {F}_{f 2} + {F}_{e 3} \\ {F}_{D} = {F}_{e 1} + {F}_{f 2} + {F}_{f 3} \\ {F}_{E} = {F}_{f 1} + {F}_{f 2} + {F}_{f 3} \\ {F}_{F} = {F}_{f 1} + {F}_{e 2} + {F}_{f 3}\end{matrix} & ( 1 )\end{matrix}$$

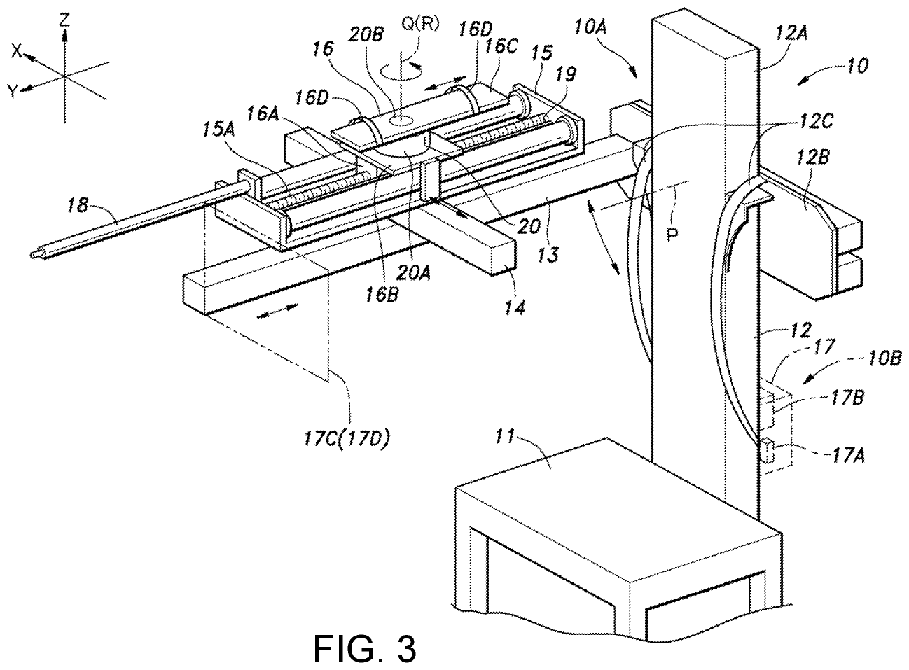

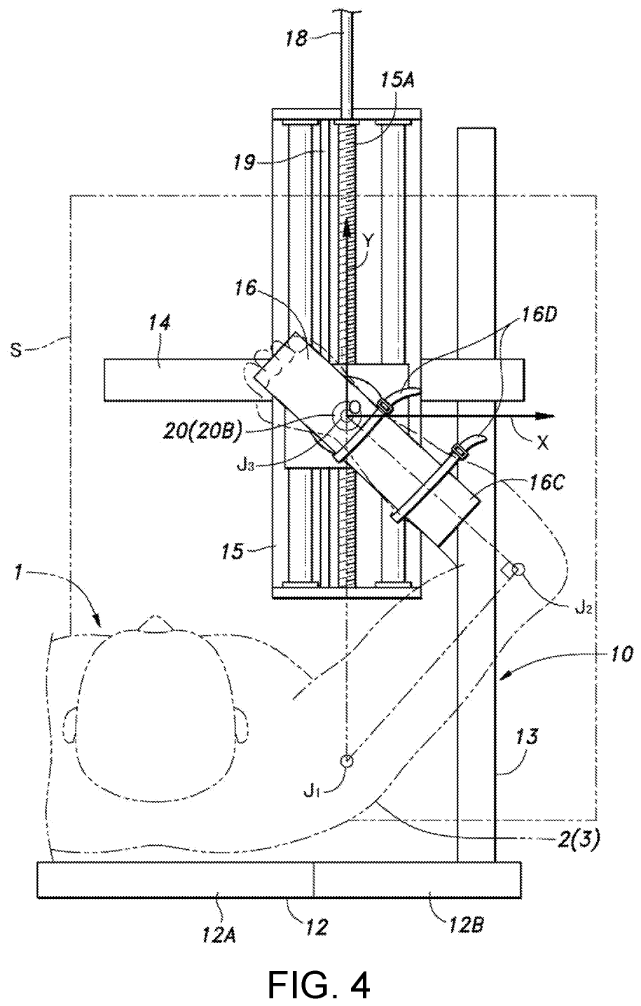

Next, referring to FIG. 3 and FIG. 4, a muscular strength characteristic evaluation device 10 for applying the muscular strength characteristic evaluation method according to the disclosure to measurement of a force exerted by the upper limb 2 will be described. As shown in FIG. 3, the muscular strength characteristic evaluation device 10 includes an acquisition device 10A (acquisition means) for acquiring outputs of a carpal joint (the free end of the second rod Lz) at two or more different velocities, and a processing device 10B for calculating a function indicating a relationship between the output and the velocity based on a data acquired by the acquisition device 10A to evaluate a muscular strength.

The acquisition device 10A includes a seating part 11, a backrest 12 joined with the rear part of the seating part 11 (see also FIG. 4), a reference arm 13 joined with the backrest 12 at an end part and extending forward, an orthogonal arm 14 supported on the upper surface of the reference arm 13, a slide unit 15 supported by the orthogonal arm 14, and a drive unit 16 supported by the slide unit 15.

The backrest 12 extends vertically and includes a base part 12A fixed to the seating part 11 and an extension part 12B fixed to the rear surface of the base part 12A. The extension part 12B has a plate shape extending in the left-right direction. The extension part 12B is joined with the rear surface of the base part 12A at a substantially central part in the left-right direction, and the left and right ends of the extension part 12B are located on the left and right outer sides of the base part 12A.

The backrest 12 is provided with belts 12C respectively on the left and right sides for respectively fixing the waist and shoulders of the subject 1 at the time of muscular strength measurement.

The reference arm 13 has a prismatic shape extending in the front-rear direction. The rear end of the reference arm 13 is joined with the front surface of the extension part 12B on the right side of the base part 12A. In this embodiment, the reference arm 13 is supported to be rotatable with respect to the backrest 12 around an axis P extending in the front-rear direction through a substantial center in the left-right direction of the backrest 12.

The orthogonal arm 14 is a member having a substantially prismatic shape and is joined with the upper surface of the reference arm 13. The orthogonal arm 14 is supported by its lower surface to be slidable along the extending direction of the reference arm 13.

The slide unit 15 includes a rail 15A extending in a predetermined direction. The slide unit 15 is supported to be rotatable on an axis Q, which is the vertical direction, with respect to the upper surface of the orthogonal arm 14, and slidable along the extending direction of the orthogonal arm 14.

The drive unit 16 includes a slider 16A which is slidably joined with the rail 15A of the slide unit 15. Accordingly, the drive unit 16 is supported by the slide unit 15 to be slidable along the extending direction of the rail 15A. A damper 18 is provided between the slide unit 15 and the drive unit 16 to apply a resistance force (damping force) to the slide movement of the drive unit 16. The resistance force of the damper 18 may be changed by a predetermined method.

The damper 18 may be of a known hydraulic type (e.g., ACE Controls, HB-28-500).

An encoder 19 is provided between the rail 15A and the slider 16A to measure a position and a velocity of the slider 16A with respect to the rail 15A. The encoder 19 may be of a known optical type (e.g., Renishaw, QUANTiC series). Taking the set position as an origin O, the encoder 19 measures the position of the slider 16A in the extending direction with respect to the rail 15A and the movement velocity of the slider 16A.

The drive unit 16 includes a base part 16B having a substantially rectangular plate shape and joined with the slider 16A, and a fixing plate 16C supported on the upper surface of the base part 16B to be rotatable on a rotation axis R extending in the vertical direction. The fixing plate 16C is a plate member having a substantially rectangular shape, and is provided with two bands 16D for fixing the subject 1 from the upper arm to the hand part. In this embodiment, the rotation axis R and the axis Q are set at the same position.

The drive unit 16 is provided with a six-axis force sensor 20 (sensor). The six-axis force sensor 20 is arranged along the rotation axis R of the fixing plate 16C and is fixed to the upper surface of the base part 16B. The six-axis force sensor 20 includes a body 20A having a substantially cylindrical shape which is opened toward the upper side, and a detection part 20B having a cylindrical shape centered on the rotation axis R and housed in the body 20A. The body 20A of the six-axis force sensor 20 is fixed to the upper surface of the base part 16B. The detection part 20B projects upward from the upper edge of the body 20A, and the upper surface of the detection part 20B is flush with the fixing plate 16C. As shown in FIG. 4, when the upper arm of the subject 1 is fixed to the fixing plate 16C, the carpal joint portion (the limb distal end J₃) of the subject 1 is set to be in contact with the upper surface of the detection part 20B.

The six-axis force sensor 20 measures forces in three axial directions applied to the upper surface of the detection part 20B and moments around each axis. Among these three axes, the direction along the extending direction of the orthogonal arm 14 and away from the subject 1 (rightward direction) is set as the X-axis, the extending direction of the reference arm 13 away from the subject 1 (forward direction) is set as the Y-axis, and the upward direction is set as the Z-axis (see FIG. 3 and FIG. 4). In other words, the six-axis force sensor 20 measures the forces in the X-axis, Y-axis, and Z-axis directions applied to the upper surface of the detection part 20B and the moments around the X-axis, Y-axis, and Z-axis. The six-axis force sensor 20 may be a known strain gauge type sensor.

As shown in FIG. 3, the processing device 10B is configured by a computer 17 including a calculation processing part 17A, such as a central processing unit (CPU), which performs calculation processing, a storage part 17B, such as a memory and a hard disk, which stores and holds information, and an input/output part 17C. In this embodiment, the input/output part 17C includes a touch panel 17D. The touch panel 17D displays buttons and input fields as appropriate to receive an input from the subject 1 and an assistant who assists the subject 1. In addition, the touch panel 17D displays texts or the like to instruct the subject 1 and the assistant and appropriately displays an evaluation result of the muscular strength.

The processing device 10B is connected to the encoder 19 and the six-axis force sensor 20 via a predetermined cable. The processing device 10B acquires the outputs from the encoder 19 and the six-axis force sensor 20 via the cable to acquire the position and the movement velocity of the slider 16A measured by the encoder 19 and the forces in the X-axis, Y-axis, and Z-axis directions and the moments around each axis measured by the six-axis force sensor 20.

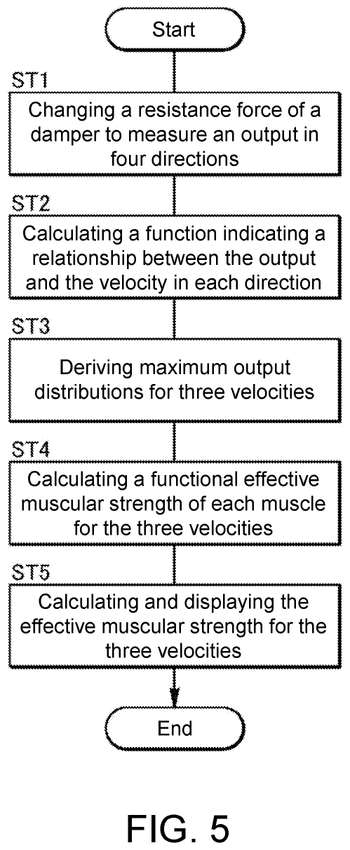

When there is a predetermined input on the touch panel 17D, the processing device 10B executes a muscular strength evaluation processing for evaluating the muscular strength of the upper limb 2 of the subject 1. Hereinafter, the details of the muscular strength evaluation processing will be described with reference to the flowchart shown in FIG. 5.

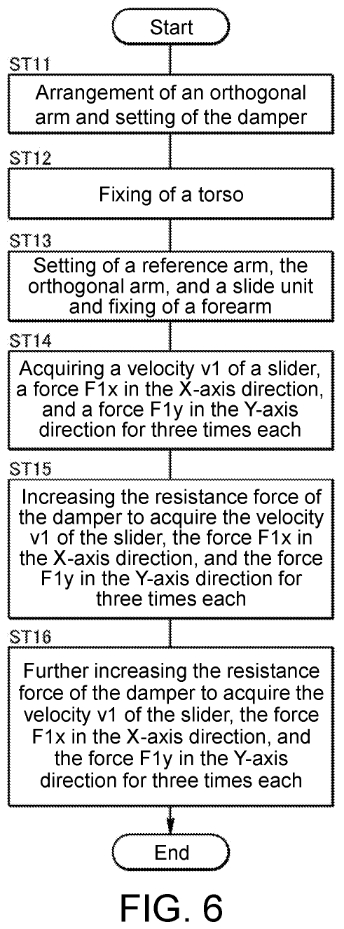

In the muscular strength evaluation processing, first, the processing device 10B performs Step ST1 (measurement step) of measuring an output exerted at the carpal joint portion when the carpal joint portion is moved at different velocities along at least four directions in a measurement plane S defined by the humerus and the radius. In this embodiment, the movement direction of the carpal joint portion is set as the forward, rearward, leftward, and rightward directions of the subject 1. In the following, the process of measuring the output exerted at the carpal joint portion (hereinafter referred to as a forward measurement process) when the carpal joint portion is moved forward will be described with reference to FIG. 6.

In the forward measurement process, the processing device 10B first instructs the subject 1 and the assistant to arrange the slide unit 15 to extend in the front-rear direction along the reference arm 13 (see FIG. 4) and set the resistance force of the damper 18 to a predetermined value (ST11). Afterwards, in the touch panel 17D, the processing device 10B receives an input from the subject 1 or the assistant indicating that the arrangement of the orthogonal arm 14 and the setting of the resistance force of the damper 18 are completed.

Upon receiving the input from the subject 1 or the assistant indicating that the setting is completed, the processing device 10B instructs the subject 1 to sit on the seating part 11 so that his/her back is along the backrest 12, and fix the torso to the backrest 12 (fixing part) by the belts 12D (ST12). Afterwards, the processing device 10B appropriately displays a button on the touch panel 17D and receives an input from the subject 1 or the assistant indicating that the fixing is completed.

Upon receiving the input indicating that the fixing is completed, the processing device 10B instructs the subject 1 and the assistant to adjust the position of the reference arm 13, so that when the upper arm (the first rod L₁) is fixed to the fixing plate 16C by moving the reference arm 13 in the vertical direction, the positions of the humerus and the radius (or the ulna) (the second rod L₂) are within the same horizontal measurement plane S. Further, the processing device 10B instructs to move the orthogonal arm 14 in the front-rear direction with respect to the reference arm 13, and while moving the slide unit 15 in the left-right direction with respect to the orthogonal arm 14, rotate the slide unit 15 appropriately to arrange and fix the rail 15A to extend in the front-rear direction in front of the shoulder joint (the first joint J₁), so that the angle formed by the upper arm (the first rod L₁) and the radius (or the ulna) (the second rod L₂) is 90 degrees, and the shoulder joint (the first joint J₁) and the carpal joint portion (the limb distal end J₃) are aligned in the front-rear direction. As a result, as shown in FIG. 4, the slide unit 15 is arranged to extend in the front-rear direction in front of the shoulder joint (the first joint J₁), and is in a fixed state with respect to the backrest 12 (ST13).

Afterwards, the processing device 10B displays an instruction to place the forearm on the fixing plate 16C and fix it with the bands 16D. Accordingly, when the subject 1 moves the carpal joint portion in the front-rear direction, the drive unit 16 slides in the front-rear direction along the rail 15A along with the movement of the carpal joint portion. The processing device 10B displays a button on the touch panel 17D as appropriate, and receives an input indicating that the setting of the positions of the reference arm 13, the orthogonal arm 14, and the slide unit 15 is completed.

Upon receiving the input indicating that the setting of the positions is completed, the processing device 10B acquires the position of the slider 16A from the encoder 19 and sets it as the origin O, and then instructs the subject 1 to take a sufficient run-up distance and apply a maximum force to move the carpal joint portion forward for three times. As the subject 1 applies a load to the carpal joint portion, the drive unit 16 moves forward along the rail 15A. At this time, since the run-up distance is sufficiently taken, the drive unit 16 moves at a substantially constant velocity when passing through the origin O, and a force F₁ₓ in the X-axis direction and a force F_{1y} in the Y-axis direction detected by the six-axis force sensor 20 correspond to the output exerted by the subject 1 when the limb distal end J₃ moves at the movement velocity of the drive unit 16. At this time, based on the force F₁ₓ in the X-axis direction and the force F_{1y} in the Y-axis direction acquired by the six-axis force sensor 20, the processing device 10B may display the direction of the output exerted by the subject 1 on the touch panel 17D. Accordingly, it is possible to confirm whether the subject 1 is exerting the force in the right direction.

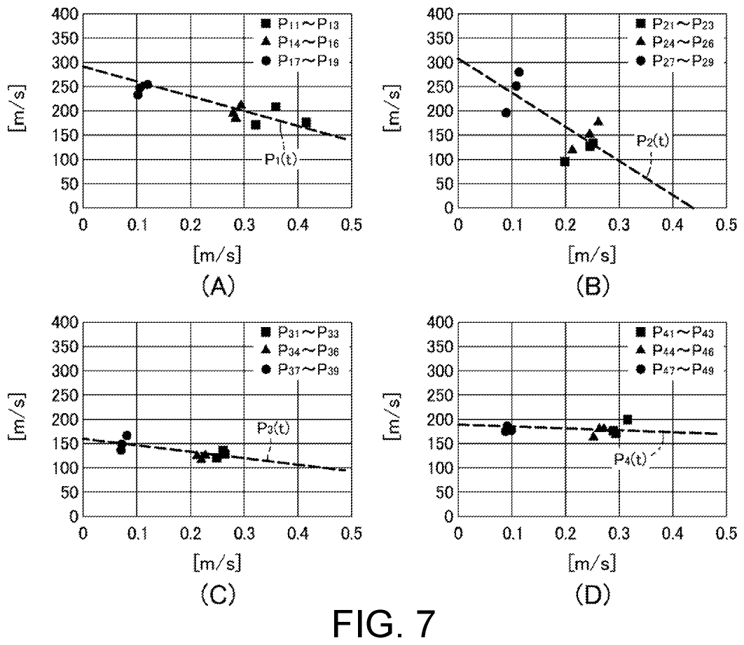

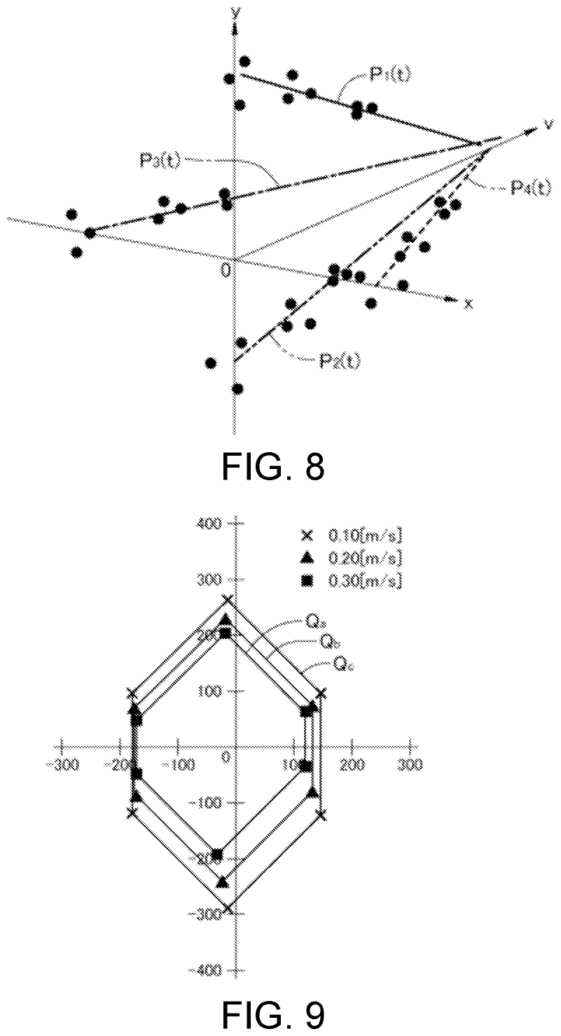

The processing device 10B acquires the position of the slider 16A from the encoder 19 and acquires a velocity v₁ of the slider 16A, the force F₁ₓ in the X-axis direction, and the force F_{1y} in the Y-axis direction for three times each when the slider 16A passes through the origin O (ST14). As a result, as shown in FIG. 7, three sets of data including three values of the velocity v₁, the force F₁ₓ in the X-axis direction, and the force F_{1y} in the Y-axis direction are acquired. Hereinafter, as shown in FIG. 8, the set of the velocity v₁, the force F₁ₓ in the X-axis direction, and the force F_{1y} in the Y-axis direction as acquired will be regarded as a point on a three-dimensional space defined by the velocity v₁, the force F₁ₓ in the X-axis direction, and the force F_{1y} in the Y-axis direction, and will be recorded as an output point P (i=1, 2, 3; “i” indicates the sequence in which the measurement with respect to the forward direction is performed).

Upon completing the measurement for three times, by increasing the resistance force of the damper 18 to perform the same measurement, the processing device 10B acquires the force F₁ₓ in the X-axis direction and the force F_{1y} in the Y-axis direction at the velocity v₁ of the slider 16A different from that in Step ST14 (ST15). More specifically, the processing device 10B instructs the subject 1 and the assistant to increase the resistance force of the damper 18. Afterwards, the processing device 10B receives an input indicating that the change to the resistance force of the damper 18 is completed. Upon receiving the input indicating that the change to the resistance force of the damper 18 is completed, the processing device 10B again instructs the subject 1 to apply the maximum force to move the carpal joint portion forward for three times. Each time the slider 16A passes through the origin O, the processing device 10B acquires the velocity v₁ of the slider 16A, the force F₁ₓ in the X-axis direction, and the force F_{1y} in the Y-axis direction, i.e., the output point (i=4, 5, 6).

Afterwards, by further increasing the resistance force of the damper 18 to perform the same measurement, the processing device 10B acquires the force F₁ₓ in the X-axis direction and the force F_{1y} in the Y-axis direction at the velocity v₁ of the slider 16A different from those in Steps ST14 and ST15 (ST16). More specifically, the processing device 10B instructs the subject 1 and the assistant to increase the resistance force of the damper 18, as in Step ST15. Afterwards, the processing device 10B instructs to move the carpal joint portion forward as in Step ST15, and acquires the velocity v₁ of the slider 16A, the force F₁ₓ in the X-axis direction, and the force F_{1y} in the Y-axis direction for three times each, i.e., P₁ᵢ (i=7, 8, 9). Upon completing the acquisition of the output point P₁ᵢ (i=1 to 9), the processing device 10B ends the forward measurement process.

Accordingly, by changing the resistance force of the damper 18 and acquiring the output point P₁ᵢ (i=1 to 9), it is possible to effectively acquire the forces F₁ₓ and F_{1y} respectively at different velocities.

After the forward measurement process is completed, the processing device 10B performs the same processing as in the forward measurement process to instruct the subject 1 to move the carpal joint portion rearward (ST14 to 16), and acquires a velocity v₂ and forces F₂ₓ and F_{2y}, i.e., an output point P₂ᵢ (1=1 to 9), when the subject 1 moves the carpal joint portion rearward for three times each under three conditions of mutually different resistance forces of the damper 18.

Afterwards, the processing device 10B performs the same processing as in the forward measurement process except for instructing, in ST14 to 16, the subject 1 to move the carpal joint portion leftward. Accordingly, the processing device 10B acquires a velocity v₃ and forces F₃ₓ and F_{3y}, i.e., an output points P₃ᵢ (i=1 to 9), when the subject 1 moves the carpal joint portion leftward for three times each under three conditions of different resistance forces of the damper 18.

Afterwards, the processing device 10B performs the same processing as in the forward measurement process except for instructing, in ST14 to 16, the subject 1 to move the carpal joint portion rightward. Accordingly, the processing device 10B acquires a velocity v₄ and forces F₄ₓ and F_{4y}, i.e., an output point R₄ᵢ (i=1 to 9), when the subject 1 moves the carpal joint portion rightward for three times each under three conditions of mutually different resistance forces of the damper 18, and the measurement step is ended. As a result, the processing device 10B acquires in-plane outputs at two or more different velocities for the forward direction, the rearward direction, the leftward direction, and the rightward direction of the free end of the upper arm. Therefore, the processing device 10B ends Step ST1 (measurement step).

As shown in FIG. 5, upon completing Step ST1, based on the in-plane outputs and the velocities in the forward direction, the rearward direction, the leftward direction, and the rightward direction as acquired in the measurement step, the processing device 10B performs Step ST2 (calculation step) of calculating a function indicating a relationship between the output and the velocity in each direction. Since the processing in the forward direction, the rearward direction, the leftward direction, and the rightward direction is the same, in the following, the processing for calculating the function indicating the relationship between the output and the velocity in the forward direction will be described, and the description for the other directions will be omitted.

Using the measured output points P₁ᵢ (i=1 to 9), the processing device 10B calculates a function P₁(t) indicating the relationship between the velocity and the force in the corresponding direction (forward direction). In other words, the processing device 10B functions as a calculation means (calculation device) which calculates a function indicating the relationship between the forces F₁ₓ and F_{1y} and the velocity v₁ in the forward direction based on the output points P₁ᵢ (i=1 to 9). In this embodiment, as shown in (A) of FIG. 7, the processing device 10B assumes that the relationship between the velocity and the force in the forward direction can be approximated by the following linear line and calculates coefficients a, b, and c based on the least squares method.

$$\begin{matrix}[ Mathematical Formula 2 ] & \\ {P}_{1} ( t ) = ( \begin{matrix}{F}_{1 x} \\ {F}_{1 y} \\ {ν}_{1}\end{matrix} ) = ( \begin{matrix}{F}_{1 x 0} + a t \\ {F}_{1 y0} + bt \\ {v}_{10} + ct\end{matrix} ) ( where {a}^{2} + {b}^{2} + {c}^{2} = 1 ) & ( 2 )\end{matrix}$$

For example, using the measured output points P₁ᵢ (i=1 to 9), the processing device 10B may calculate L and N, respectively.

$$\begin{matrix}[ Mathematical Formula 3 ] & \\ \{ \begin{matrix}L = \frac{Cov ( {F}_{1 x} , {v}_{1} )}{{s}_{v 1}^{2}} \\ N = \frac{Cov ( {F}_{1 y} , {v}_{1} )}{{s}_{v 1}^{2}}\end{matrix} & ( 3 )\end{matrix}$$

In the formula, Cov(F₁ₓ, v₁) represents a covariance of Fix and the velocity v₁, Cov(F_{1y}, v₁) represents a covariance of F_{1y} and the velocity v₁, and sᵥ₁² represents a variance of the velocity v₁. Afterwards, by calculating F₁ₓ₀, F_{1y0}, v₁ᵥ₀, a, b, and c using Formula (4) below, the processing device 10B may acquire the function indicating the relationship between the velocity and the force represented by Formula (2).

$$\begin{matrix}[ Mathematical Formula 4 ] & \\ \{ \begin{matrix}{F}_{1 x 0} = {μ}_{{F}_{1 x}} \\ {F}_{1 y 0} = {μ}_{{F}_{1 y}} \\ {v}_{10} = {μ}_{{v}_{1}} \\ a = \sqrt{\frac{{L}^{2}}{{L}^{2} + {N}^{2} + 1}} \\ b = \sqrt{\frac{{N}^{2}}{{L}^{2} + {N}^{2} + 1}} \\ c = \sqrt{\frac{1}{{L}^{2} + {N}^{2} + 1}}\end{matrix} & ( 4 )\end{matrix}$$

In the formula, μ_{F1x}, μ_{F1y}, and μᵥ₁ represent a mean of the force F₁ₓ, a mean of the force F_{1y}, and a mean of the velocity v₁, respectively.

Upon completing the acquisition of the function P₁(t) indicating the relationship between the velocity and the force in the forward direction, the processing device 10B performs the same processing for each of the rearward direction, the leftward direction, and the rightward direction, and as shown in FIG. 7 and FIG. 8, acquires linear functions P₂(t), P₃(t), and P₄(t) indicating the relationship between the velocity and the force for the four directions. Upon completing the acquisition of the functions in the four directions, the processing device 10B ends Step ST2.

Upon completing Step ST2, as shown in FIG. 5, the processing device 10B performs Step ST3 (hereinafter referred to as a creation step) of respectively deriving maximum output distributions Qₐ, Q_{b}, and Q_{c} in a hexagonal shape at the different velocities. More specifically, first, using the acquired functions Pᵢ(t) (i=1 to 4), the processing device 10B acquires an output (F₁ₓ, F_{1y}) in the forward direction, an output (F₂ₓ, F_{2y}) in the rearward direction, an output (F₃ₓ, F_{3y}) in the leftward direction, and an output (F₄ₓ, F_{4y}) in the rightward direction at each of three predetermined velocities vₐ, v_{b}, and v_{c}.

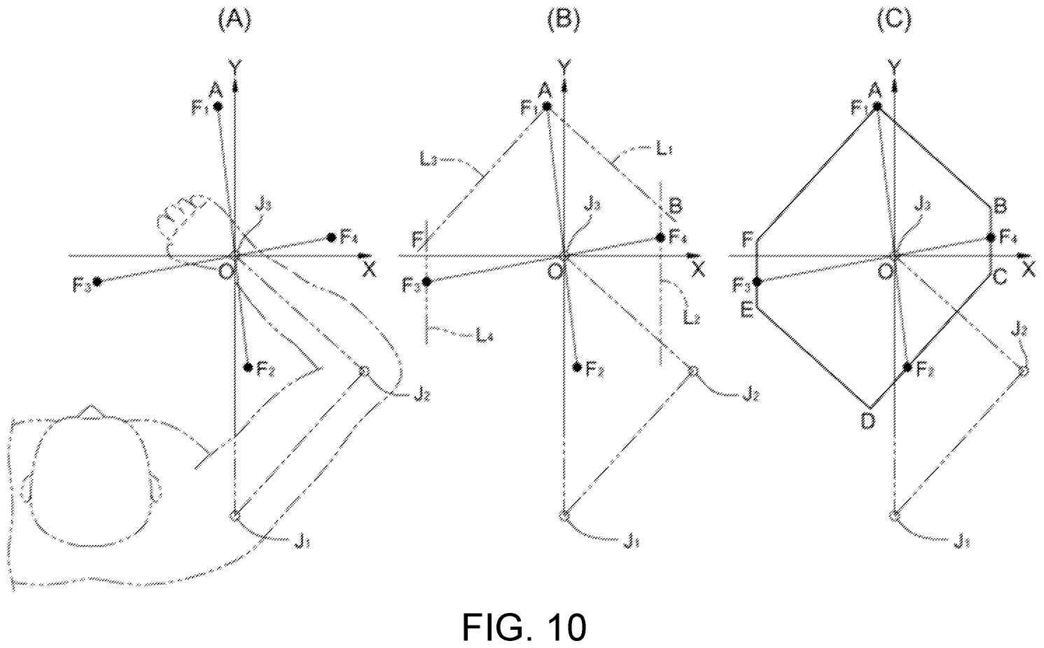

Next, using the outputs in the four directions, i.e., the forward direction, the rearward direction, the leftward direction, and the rightward direction, respectively corresponding to the three velocities vₐ, v_{b}, and v_{c}, the processing device 10B obtains the maximum output distributions Qₐ, Q_{b}, and Q_{c} in a hexagonal shape, respectively, based on a known four-point measurement method, as shown in FIG. 9.

Hereinafter, referring to FIG. 10, the derivation of the maximum output distribution based on the four-point measurement method will be briefly described. The following description will assume the output (F₁ₓ, F_{1y}) (hereinafter, F₁) in the forward direction, the output (F₂ₓ, F_{2y}) (hereinafter, F₂) in the rearward direction, the output (F₃ₓ, F_{3y}) (hereinafter, F₃) in the leftward direction, and the output (F₄ₓ, F_{4y}) (hereinafter, F₄) in the rightward direction at the velocities for deriving the maximum output distributions. The processing device 10B first sets F₁ as a vertex A of the hexagon ((A) of FIG. 10). Next, the processing device 10B sets as a vertex B an intersection point between a straight line L₁ passing through the vertex A and parallel to a straight line connecting the joints J₂ (elbow joint) and J₃ (carpal joint portion), and a straight line L₂ passing through F₄ and parallel to a straight line connecting J₁ (shoulder joint) and J₃ (carpal joint portion). Similarly, the processing device 10B sets as a vertex F an intersection point between a straight line L₃ passing through the vertex A and parallel to a straight line connecting J₁ and J₂, and a straight line L₄ passing through F₃ and parallel to a straight line connecting J₁ and J₃ ((B) of FIG. 10).

Next, an intersection point between a straight line L₅ passing through F₂ and parallel to a straight line connecting J₁ and J₂, and the straight line L₂ is set as a vertex C. Afterwards, a point separated by a length (l) of a line segment AF from the vertex C on the straight line L₅ is set as a vertex D, and an intersection point between a straight line L₆ passing through the vertex D and parallel to a straight line connecting J₂ and J₃ and, and the straight line L₄ is set as a vertex E ((C) of FIG. 10). At this time, a vector AF having the vertex A as the start point and the vertex F as the end point and a vector CD having the vertex C as the start point and the vertex D as the end point are equal to each other, and a vector AB having the vertex A as the start point and the vertex B as the end point and a vector DE having the vertex E as the start point and the vertex D as the end point are equal to each other. By obtaining the vertices A to F in this manner, a maximum output distribution in a hexagonal shape which connects the vertices can be obtained.

However, in this embodiment, since J₃ is located in front of J₁, both L₂ and L₄ are parallel to the Y-axis, and since the angle of the elbow joint is set to 90 degrees, L₁ and L₃ are respectively straight lines extending at an angle of 45 degrees in the XY plane.

After Step ST3, the processing device 10B performs Step ST4 (contribution amount calculation step) of calculating functional effective muscular strengths F_{f1}, F_{f2}, F_{f3}, Fₑ₁, Fₑ₂, and Fₑ₃ at each of the three velocities vₐ, v_{b}, and v_{c}. More specifically, the processing device 10B calculates the functional effective muscular strengths F_{f1}, F_{f2}, F_{f3}, Fₑ₁, Fₑ₂, and Fₑ₃ of the muscles respectively from the maximum output distributions Qₐ, Q_{b}, and Q_{c} corresponding to the velocities vₐ, v_{b}, and v_{c}, respectively, based on Formula (1). At this time, in addition to the hexagon ABCDEF and Formula (1), the processing device 10B may calculate the functional effective muscular strengths F_{f1}, F_{f2}, F_{f3}, Fₑ₁, Fₑ₂, and Fₑ₃ of the muscles by also setting an appropriate numerical value as the ratio of the magnitudes of two antagonistic muscular strengths, for example, |F_{f1}|/(|F_{f1}|+|Fₑ₁|).

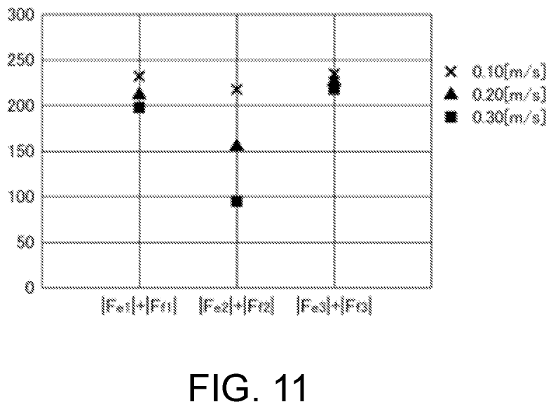

After Step ST4, the processing device 10B performs Step ST5 (calculation step) of calculating and displaying effective muscular strengths of the muscle pairs at the three velocities vₐ, v_{b}, and v_{c} based on the calculated functional effective muscular strength of each muscle. More specifically, the processing device 10B calculates an effective muscular strength |Fₑ₁|+|F_{f1}| of the first antagonistic monoarticular muscle pair e₁ and f₁, an effective muscular strength |Fₑ₂|+|F_{f2}| of the second antagonistic monoarticular muscle pair e₂ and f₂, and an effective muscular strength |Fₑ₃|+|F_{f3}| of the antagonistic biarticular muscle pair e₃ and f₃ at each of the three velocities vₐ, v_{b}, and v_{c}. Afterwards, the processing device 10B displays the result as a graph on the touch panel 17D as shown in FIG. 11. Upon completing the display, the processing device 10B ends the muscular strength evaluation processing.

Next, the effect of the muscular strength evaluation method configured in this manner will be described. FIG. 7 shows (A) the relationship between the velocity v₁ and the magnitude of the forward-direction component of the force of the forward-direction outputs P₁₁ to P₁₉, (B) the relationship between the velocity v₂ and the magnitude of the rearward-direction component of the force of the rearward-direction outputs P₂₁ to P₂₉, (C) the relationship between the velocity v₃ and the magnitude of the leftward-direction component of the force of the leftward-direction outputs P₃₁ to P₃₉, and (D) the relationship between the velocity v₄ and the magnitude of the rightward-direction component of the force of the rightward-direction outputs P₄₁ to P₄₉, respectively, as measured according to the muscular strength evaluation method. In (A) to (D) of FIG. 7, the functions P₁(t) to P₄(t) indicating the relationship between the velocity and the force obtained in each direction are shown by broken lines. As shown in (A) to (D) of FIG. 7, the points obtained by the measurement are generally located on the broken line. Therefore, when considering only the sections near the velocities measured in the experiment, it can be confirmed that the outputs in the four directions (i.e., the forward, rearward, leftward, and rightward directions) can be approximated by the linear function of the velocity. In addition, by using the linear function, an approximate expression indicating the relationship between the output and the velocity can be easily calculated. Moreover, since the output is represented by the linear function of the velocity, the output at each velocity can be easily calculated.

As shown by the broken lines in (A) to (D) of FIG. 7, by performing approximation by the least squares method, the processing device 10B can acquire from the measurement data the functions P₁(t), P₂(t), P₃(t), and P₄(t) indicating the relationship between the output and the velocity in each of the four directions (i.e., the forward, rearward, leftward, and rightward directions). As a result, by using these functions, the processing device 10B can acquire the outputs in the four directions at any velocity.

By using the function showing the relationship between the output and the velocity, the processing device 10B can obtain the maximum output distribution at any velocity based on the four-point measurement method. FIG. 9 shows the maximum output distributions Qₐ, Q_{b}, and Q_{c} at each of the velocities vₐ=0.1 m/s, v_{b}=0.2 m/s, and v_{c}=0.3 m/s, as acquired by the processing device 10B of this embodiment.

As described above, in the muscular strength evaluation method according to the disclosure, the maximum output distribution for any velocity can be obtained by performing the four-point measurement method using the function showing the relationship between the output and the velocity. Accordingly, by obtaining each effective muscular strength at any velocity, it is possible to evaluate each effective muscular strength according to the velocity.

FIG. 11 shows the effective muscular strength |Fₑ₁|+|F_{f1}| of the first antagonistic monoarticular muscle pair e₁ and f₁, the effective muscular strength |Fₑ₂|+|F_{f2}| of the second antagonistic monoarticular muscle pair e₂ and f₂, and the effective muscular strength |Fₑ₃|+|_{f3}| of the antagonistic biarticular muscle pair e₃ and f₃ at each of the velocities vₐ, v_{b}, and v_{c}, as calculated based on the maximum output distributions Qₐ, Q_{b}, and Q_{c}. Based on FIG. 11, it can be confirmed that the decrease rate of the effective muscular strength |Fₑ₂|+|F_{f2}| of the second antagonistic monoarticular muscle pair e₂ and f₂ (i.e., the effective muscle group which acts on the elbow joint) due to the increase in the velocity is larger than the others. From this result, it can be understood that the ratio of slow muscles in the muscle group acting on the elbow joint is smaller than that in other muscle groups. In other words, the muscular strength evaluation method according to the disclosure may be used as an index for knowing the ratio between fast muscles and slow muscles. Accordingly, it is possible to identify the muscles to be strengthened according to the competition. Therefore, more effective training may be carried out by applying the muscular strength evaluation method according to the disclosure to muscular strength evaluation for rehabilitation and sports.

Although the specific embodiment has been described above, the disclosure may be widely modified without being limited to the above embodiment. While the processing device 10B acquires the maximum output in the four directions in Step ST1, the processing device 10B is not limited thereto. The processing device 10B may be configured in any manner in Step ST1 as long as the maximum output required for obtaining the maximum output distribution in a hexagonal shape can be acquired in Step ST3. More specifically, for example, in Step ST1, the processing device 10B may acquire the maximum output in five or more directions to acquire the maximum output distribution in a hexagonal shape, or may acquire the maximum output at each predetermined angle in the circumferential direction to acquire the maximum output distribution in a hexagonal shape.

In the above embodiment, while output points in the four directions are acquired and the maximum output distributions in a hexagonal shape at each velocity are obtained, the embodiment is not limited thereto. For example, the processing device 10B may be configured to acquire the output at the origin O of the carpal joint portion when the carpal joint portion is moved at two or more different velocities in one predetermined direction or multiple directions, and calculate a function indicating the relationship between the force and the velocity in that direction. The function outputted by the processing device 10B may be a linear function, and may be, for example, the same as Formula (2). At this time, the processing device 10B may display a graph similar to that in (A) of FIG. 7 indicating the change in the output with respect to the velocity on the touch panel 17D. Accordingly, the subject 1 and the assistant can evaluate the velocity dependence of the muscular strength according to its slope.

In the above embodiment, while the velocity is changed by the resistance force set by the damper 18 to acquire the output, the embodiment is not limited thereto. For example, a motor which moves the drive unit 16 with respect to the slide unit 15 at a predetermined velocity may be provided. Accordingly, since the movement velocity of the drive unit 16 may be set, the movement velocity of the carpal joint portion can be set in a more fine-tuned manner. As a result, it is possible to measure the velocity dependence of the maximum output in greater details.

Nonetheless, in this embodiment, the resistance force applied to the carpal joint portion is applied by the damper 18, and the velocity of the carpal joint portion is changed by changing the resistance force. Therefore, it is easy to change the velocity of the carpal joint portion. Also, compared to the case where the carpal joint portion is moved by a motor, since the subject 1 may move the upper arm by his/her own will, it is possible to reduce the anxiety which may be caused to the subject 1 at the time of muscular strength evaluation.

In the above embodiment, while the muscular strength characteristic evaluation method is used to evaluate the characteristic of the muscular strength of the upper limb 2 on the right side of the subject 1, the method is not limited to the upper limb 2 on the right side of the subject 1, and the method may be applied to the upper limb 2 on the left side of the subject 1, or the lower limb on either the left or right side of the subject 1. Further, in the above embodiment, while the measurement plane S is set to be substantially horizontal, the disclosure is not limited thereto, and for example, the measurement plane S may be set to be substantially vertical. In addition, the muscular strength evaluation measurement method may be used to evaluate the muscular strength of animals such as horses, cows, dogs, and the like.

What is claimed is:

1. A muscular strength characteristic evaluation method, which evaluates a muscular strength characteristic of a limb comprising a first rod having a base end supported by a first joint, and a second rod supported by a free end of the first rod via a second joint, the muscular strength characteristic evaluation method comprising:
a step of moving a free end of the second rod at two or more different velocities in a predetermined direction, and respectively measuring an output at the free end of the second rod at a predetermined position;
a step of calculating a function indicating a relationship between the output and the velocity in the direction based on the output and the velocity; and
a step of evaluating the muscular strength characteristic based on the function.

2. The muscular strength characteristic evaluation method according to claim 1, wherein
in the step of measuring the output, the direction is set to at least four different directions in a plane defined by the first rod and the second rod,
in the step of calculating the function indicating the relationship between the output and the velocity, the function is calculated with respect to each of the directions, and
the step of evaluating the muscular strength characteristic comprises a step of calculating the output in each of the directions at the predetermined velocity by using the function, and creating a maximum output distribution in a hexagonal shape corresponding to contributions of each muscle of a muscle group model comprising a first antagonistic monoarticular muscle pair straddling the first joint, a second antagonistic monoarticular muscle pair straddling the second joint, and an antagonistic biarticular muscle pair straddling the first joint and the second joint.

3. The muscular strength characteristic evaluation method according to claim 2, wherein the step of evaluating the muscular strength characteristic further comprises a step of calculating a contribution amount of each muscle of the muscle group model from the maximum output distribution.

4. The muscular strength characteristic evaluation method according to claim 1, wherein a linear function is used as the function indicating the relationship between the output and the velocity.

5. The muscular strength characteristic evaluation method according to claim 1, wherein the free end of the second rod is moved at the two or more velocities by applying multiple resistance forces to the free end of the second rod.

6. A muscular strength characteristic evaluation device, which evaluates a muscular strength characteristic of a limb comprising a first rod having a base end supported by a first joint, and a second rod supported by a free end of the first rod via a second joint, the muscular strength characteristic evaluation device comprising:
an acquisition means respectively acquiring an output of a free end of the second rod at two or more different velocities in a predetermined direction at a predetermined position; and
a calculation means calculating a function indicating a relationship between the output and the velocity in the direction based on the output and the velocity.

7. The muscular strength characteristic evaluation device according to claim 6, wherein the calculation means uses a linear function as the function indicating the relationship between the output and the velocity.

8. The muscular strength characteristic evaluation device according to claim 6, wherein the acquisition means comprises:
a fixing part fixing a subject;
a slide unit fixed to the fixing part;
a drive unit provided on the slide unit to be slidable in the direction;
a sensor provided on the drive unit and detecting the output; and
a damper provided between the slide unit and the drive unit,
wherein a resistance force of the damper is variable so that the free end of the second rod is movable at the two or more velocities by applying multiple resistance forces to the free end of the second rod.
